(19) United States
(12) Patent Application Publication
(10) Pub. No.: US 2007/0172424 A1
(43) Pub. Date: Jul. 26, 2007

(54) Enabling drug adherence through closed loop monitoring & communication

(76) Inventor: Mark Costin Roser, Hebron, CT (US)

Correspondence Address: JANUS MEDICAL INSTRUMENTS, INC., 13303 CHAMPION FOREST DRIVE #5, HOUSTON, TX, 77069, US

(21) Appl. No.: 11/698,642

(22) Filed: Jan. 25, 2007

Related U.S. Application Data
(60) Provisional application No. 60/761,899, filed on Jan. 26, 2006.
(60) Provisional application No. 60/861,035, filed on Nov. 27, 2006.

(51) Int. Cl. G06Q 50/00; A61K 49/00

(52) U.S. Cl. 424/9.1; 705/3

(57) ABSTRACT

A method is described for measuring the blood concentration of a medicament through the introduction of a tracer compound. The measurement of the blood concentration of the tracer will yield a result that will enable a prediction of the blood concentration of the medicament. The method further describes ways to utilize the results for monitoring adherence to the medicament and modifying behavior to help patients boost compliance.

### CROSS-REFERENCE TO RELATED APPLICATIONS

This document follows upon Provisional 60/761,899 dated Jan. 26, 2006 and Provisional 60/861,035 dated Nov. 27, 2006, both by same inventor, Mark Costin Roser.

### STATEMENT REGARDING FEDERALLY SPONSORED RESEARCH OR DEVELOPMENT

N/A

### REFERENCE TO SEQUENCE LISTING, A TABLE, OR A COMPUTER PROGRAM LISTING COMPACT DISC APPENDIX

N/A

### BACKGROUND OF THE INVENTION

There is a longstanding problem in the healthcare field which has not yet been sufficiently addressed. This problem is the lack of many patients' ability to take their medication appropriately. It is widely known that patients struggle with both adherence (remembering to take one's medicine at the right time and on appropriate day to day schedule) as well as compliance (continuing to take medications for the entire duration of the treatment protocol which may be months or years for chronic conditions).

Many life-threatening diseases are chronic and require taking medications throughout the life of the patient; these include cardiovascular, viral, metabolic, ophthalmologic and many others. Of particular issue are infectious diseases such as HIV that require anti-viral and anti-retro-viral therapy to sustain the life of the patient, prevent re-transmission and reduce the likelihood of worsening the severity of the untreated virus.

When someone feels discomfort, and finds relief through a pharmaceutical agent, there is an inherent and obvious reward to taking the medication at the appropriate time and dosage.

The ability for patients to notice a beneficial physical or neurological result associated with taking their medication stands in sharp contrast to long-term drug therapies for chronic conditions. In such circumstances, patients do not feel an immediate physical or neurological result from taking their medication at the appropriate dose and time. Any untoward effects may not become noticeable for months or years.

Patients do, however, can feel the physical discomfort of taking their medication (ie: the discomfort of swallowing pills or any side-effects associated with the drug), can sense the frustration associated with remembering what pills to take at the right times and can experience the psychological consequences of worrying whether they remembered to take their medication the previous day or not.

The reality is that patients in long-term drug therapy experience a large number of negatives while not experiencing many noticeable benefits other than the internal knowledge that they are doing the right thing to stay healthy.

This result is a high percentage of patients who do not adhere/comply with their drug therapy protocol. A variety of studies show that for chronic illness sufferers, as many as 50% or more patients do not continue with their course of drug therapy past the 90 to 180 day period after the initial prescription is provided.

Implications of patient non-compliance extend far beyond the immediate impact to the patient:
Patients who are not adherent/compliant often do not get the benefit they need from the drug they are taking
The consequence is that the prescribing doctor may incorrectly assume the patient's lack of improvement is the result of a lack of drug efficacy instead of the lack of adherence/compliance
The secondary consequence to the said misinterpretation of drug efficacy is that doctors may either increase the drug dosage or switch to a different therapeutic agent
The tertiary consequence of increasing the drug dosage may then increase the side effects associated with the drug and put the patient at risk when he or she returns to taking their medication
Another tertiary consequence of switching to a different therapeutic agent may be an increased cost of medications (ie: assuming that generic solution did not work and transitioning to a non-generic medicine at a higher price)
The health-care payers suffer because their insureds are at higher risk for more complicated and expensive future treatment (ie: a non-compliant cardiac patient may require surgery if they are not compliant with their statin)
The pharmaceutical companies suffer because more than 50% of prescriptions written for their drug go unfilled, leaving revenues low and thereby unable to support the development of new drugs

A variety of methods have been attempted that promise to help improve the situation.

The traditional approach involves a doctor prescribing a medication, and then asking them at their follow-up visit whether they took their medication as directed. Based upon the patient's response, the doctor makes his/her care decision.

This traditional approach may be augmented by testing the patient bodily fluids to detect the presence of the drug at the appropriate levels. This approach is rarely used, except in very restricted settings such as some pharmaceutical clinical trials. The reason it is rarely used is that the time, technology and money required to sample bodily fluids for active drug concentrations is significant. It has been considered cost and time prohibited. For example, few clinical offices or hospitals even have the drug detection equipment to do this type of procedure. The approach is also lacking validity across weeks/months without testing on a near daily basis. Just because a patient's blood level was tested “good” on Monday does not mean that the patient was “good” the prior Friday.

Other methods have been proposed as a surrogate to monitoring. For example, sensor-enabled pill bottle caps have been integrated into various scenarios that detect whether a patient opened their pill bottle at an appropriate time each day. However, there is no way to understand whether the patient ingested the medicine or simply flushed it down a sink after they opened the bottle.

Other incentive programs have also been proposed that would provide gifts for patients who stayed compliant with their drug protocol. This approach will likely have strong benefits to patients on long-term care, such as adolescents and young adults on anti-retroviral HIV therapy.

Incentives might include the use of a cell-phone, a hand-held video game (ie: game boy type device, x-box type device or other gaming platform), music downloads to a digital music enabled device (ie: i-pod or other media player), personal digital assistant, food, baby food, etc. Patients may be provided these incentives when they are in compliance.

When patients fall out of compliance, the ability to receive the incentive would be suspended or retracted. For example, if the patient is provided with a game system such as x-box, they would have use of a particular game software for a limited number of minutes following each successful compliance test. Thus, this approach contains both positive feedback, by supplying access to the incentive and negative feedback when patients fall out of compliance by restricting access to the incentive.

However, such incentive programs are currently limited by the trust that the program administrator has in the patient's honest reply about their compliance.

Thus, the field of monitoring patient adherence/compliance has a critical gap in being able to identify a means of detecting blood level concentrations of active drugs in a manner that is:
Able to be performed on a regular schedule (once a week or more frequently)
Able to be performed at a reasonable cost (similar or less cost than the drug itself)
Able to provide feedback rapidly (without having to mail a sample to a remote testing facility)
Able to communicate results back to the professional caregiver accurately (with barriers to possible obfuscation by the patient)

### BRIEF SUMMARY OF THE INVENTION

A novel system is described for automating the monitoring of a patients adherence/compliance to a medication in such a way as to enable “closed loop” communications between a patient and his or her caregivers in such a way that it is able to be performed regularly, at a reasonable cost, rapidly and able to communicate accurately.

### BRIEF DESCRIPTION OF THE SEVERAL VIEWS OF THE DRAWING

N/A

### DETAILED DESCRIPTION OF THE INVENTION

Terms used in this document, AKA denotes terms used interchangeably:

Interactive Communication Device: (AKA “ICD”) A device such as a cell phone, game system, music player, personal digital assistant, or stand alone compliance enhancement unit that comprises

Pharmaceutical adherence: (AKA “Adherence”, “Adherence/Compliance”) The ability of a patient who is prescribed a medicament to ingest that medicament according to the time, quantity and frequency directed by a physician or caregiver. Lack of adherence is characterized by missing a dose of medicine or delay in ingesting ones medicine.

Active drug compound: A prescribed medicament designed for ingestion.

Administration: (AKA: Ingestion, consumption) The integration of a medicament into the bloodstream. Ie: through oral consumption, injection, transdermal application, etc.

Tracer: (AKA: Tracer Compound, Tracer/Analyte) An ingestible compound that can be detected through cost-effective and rapid means.

Bodily fluid: Blood, urine, saliva, breath.

Sensor: A device and necessary hardware/software, interconnections and algorithms needed to measure a bodily fluid in order to detect the blood-level concentration of a particular compound in question.

Pharmacokinetic Reading: (PK) The measurement of a drug's concentration in the blood over a given time as influenced by Absorption, Distribution, Metabolism & Excretion (ADME)

Patient: (Aka: user) The person who is on a drug protocol.

Physiological data: Data concerning one's health measurements such as pulse, weight, body temperature, blood sugar level, body mass index, etc.

Psychological data: Data concerning one's mental state, such as mood, feelings of depression, feelings of anxiety, suicidal thoughts, etc.

Behavioral data: Data concerning one's actions, such as the time of most recent meal, exercise level, time of most recent ingestion of a medicament, etc.

Communicatively coupled: The connection of one electronic device to another electronic device, through various means that might include wired signal, wireless signal, or the sharing of a common code that a user might read from one device and manually enter into another device.

Network server: A computer based hardware and software device that is able to communicate with a plurality of remote devices, and that also has sufficient computational and storage capability to manage a variety of storage and analytical tasks.

Behavior modification system: Implies a computerized system that has sufficient computational design a ability that it is able to establish a logical decision tree that can interpret the reported behaviors of a patient, compare them to an desired state, and deliver a response that helps a patient make better health choices.

Interactive sequence of questions: Is a set of questions that can be delivered through an I/O device that ask a patient to consider a question and answer with a response. For example: “Have you taken your medication? Press 1 for yes and 2 for no. Do you have feelings of depression right now? Press 1 for no feelings of depression, 5 for moderate feelings of depression and 9 for high level of feelings of depression.”

In this invention, an interactive communication device (ICD) is utilized to provide a patient and his or her caregiver a displayed value that allows a determination of the pharmaceutical adherence to a drug protocol.

This method starts with administering the medicament to the patient, which can simply be accomplished by traditional pill-bottle Rx labeling, or more conveniently, through an alarm/reminder provided by the ICD. In a preferred embodiment—the ICD provides a chime that audibly sounds along with a visual display that indicates what medicine to ingest at the specified time.

The next step is acquiring data associated with the administration of the medicament. This can be accomplished by a variety of means—through interactive Q&A with the patient (for example “press one if you took your medicine”), through indirect activity sensing (for example, detecting whether a patient removed the top of their pill bottle by means of a pill-top bottle opening sensor) or by detecting the presence of the drug in the bloodstream (for example, by a sensor reading).

The next step is to transmit the data associated with the administration to an ICD. This data transmission may be integral within the ICD (for example, a keypad entry using the ICD keypad) or it may be connected in some way (for example, an upload of results through electronic, optical, manual I/O means, etc)

The next step is displaying the data to enable a determination of the pharmaceutical adherence of the patient to the administered medicament. This reading may be a simple blood level concentration that must be cross-referenced to ADME/PK charts or it may be an interpreted result that states Adherent: Yes/No.

The ability for an ICD to display a patient's adherence information related to their drug protocol is a vital tool that is not currently available and requires a great expense of time and money. This invention can be of tremendous value to patients, doctors, payers, and the whole healthcare value chain.

For example, knowing a real-time adherence status will allow physicians to greatly improve their ability to understand whether a patient's slow recovery is ascribed to poor drug efficacy or poor adherence and make the appropriate follow-through Rx decision.

It will also enable pharma companies to reduce the size of clinical trials (reducing the amount of overpowering the cohort size) by improving subject monitoring and still maintain accuracy.

It will also enable patients to adjust and improve their behavior based upon frequent reminders and the presence of a visible electronic “watchdog” over their adherence.

Current means of solving this problem require non real-time means and high cost, and as such do not lend themselves to providing a solution for general healthcare that is so desperately needed.

In preferred embodiments, the inventor will show how this invention can be commercialized in various configurations. These will not be the only embodiments that those in the art can configure, but will be presented to help make a clear case.

### Preferred Embodiments

One of the prime issues with real-time adherence monitoring is the inability to trust patient-entered data or electronically reported ingestions which both are subject to possible trickery by the patient or even inadvertent discharge through vomiting. Testing the blood-level concentration of a drug is the only way to truly understand adherence.

But most drugs cannot be sensed (re: blood level concentration) without elaborate testing equipment. By administering a medicament together (ie: mixed, connected, commingled, combined, etc) with a tracer, one can make an assumption that they both will enter the bloodstream. And, then, if one can measure the concentration of the tracer in the blood, one can then calculate a predicted concentration of the medicament in the blood.

How is this done? If one has PK data for both the medicament and the tracer, and knows the relative amount of medicament relative to the amount of tracer, then one can make a reasonable correlation between the medicament blood level concentrations over time (PK) versus the tracer blood level concentrations over time (PK). This becomes cumbersome if the half-lives are radically different, but quite easy if the PK curves are similar.

And, for the purposes of this discussion of judging a patient's state of adherence, one can use the time since administration and develop a range of anticipated blood level concentrations of the tracer that accommodates variations in ADME & phenotype. Using this anticipated range, the results of an actual sensor result of a person's blood concentration (at a given time after administration) can be analyzed to judge if the reading is within bounds of the range or outside the range. And if the tracer is outside the anticipated range of concentration, one can make a reasonable judgment that the patient had issues with adherence of the medicament, and if it is inside the range, one can make a reasonable judgment that the patient was adherent.

Through this commingling of a known quantity of tracer analyte with a known quantity of active drug, the analyte would be consumed at a known dose and frequency that is directly correlated to the dose and frequency of the active drug. Detection/analysis of the tracer/analyte levels within the patient's body would then provide a mathematically correlated surrogate for understanding the patient's compliance with their dosing regiment.

In one preferred embodiment, at least one medicament is administered together with at least one tracer. In this way, the ICD can detect the tracer and make a more accurate representation of a patient's adherence.

In a more preferred embodiment, the ICD would use at least one sensor to detect the tracer in the bodily fluid of the patient.

In a further preferred embodiment, the bodily fluid would include blood, saliva, breath or urine. And in an alternative preferred embodiment, the sensing of the bodily fluid would be performed non-invasively (ie: through breath, saliva, urine analysis or external blood condition sensing such as optically).

With the cost of Internet, phone & wireless transmission of data being easy and inexpensive, one can easily see the benefit of another preferred embodiment transmitting the results of the ICD adherence reading to a network server. This would allow the result to be stored and then analyzed over time. With sufficient data points, one could create a medicament concentration model for the patient as well as an entire population.

This medicament concentration model would enable proper attention given from user to user. For example, if one patient has consistently erratic readings over time, the model could flag the user for intervention and coaching to help him/her improve their habits.

In another preferred embodiment, the ICD would be capable of asking more in-depth questions relating to how a person is feeling, their mood, how recently they ate, whether their meal was heavy or not, what their level of activity is, whether they are experiencing any effects or side-effects, etc. These may fall in any of the buckets known as physiological data, psychological data, and behavioral data. This data could be incorporated into the medicament concentration model as a means to gain further insight into a patient's behavior or health. This information could in-turn be used to gain further insight into clinical trials (for example, the patient's psychological mood worsened when they forgot their medication) or it could be used to adjust dosing (for example, if you are still in pain, you may take another pill).

A situation for use of such inventions could be visualized with a hypothetical scenario of a patient we can call “Jane”. Jane is HIV positive and is enrolled in a community program that provides her with free anti-retroviral medicine. As part of this program, Jane is given a prescription for her drug, a supply of the drug, an ICD and an introductory lesson in using the ICD. She is told that she will receive free drug as long as she is adherent, and may risk losing the supply if she is not adherent. Each morning, Jane is given an alarm together with a text message “take one blue/white capsule in the next hour, and dial *999 after you have taken your medicine”. Jane takes the medicine a half-hour later, and then dials *999 into the system. Two hours hence, an alarm sounds and a message “take sensor reading within the next hour, press *888 to start the test. 20 minutes later, Jane presses *888 and completes the sensor test (ie: saliva or breath test). The ICD calculates the tracer concentration and analyzes it against the expected PK for the 1 hour and 20 minutes following ingestion and makes a judgment whether Jane did in-fact take the drug as promised. The results are displayed to Jane and uploaded to a central network that monitors hundreds of people in the HIV care program and alerts a case worker to her status.

### Interaction Models

In these embodiments, we focus on the interaction between the patient and a medicament concentration model.

In such an embodiment, a medicament would be administered to a patient and the ICD would be able to acquire data (plural) associated with the ingestion, and then communicate this to a network server which would integrate the data into a medicament concentration model. In this way, some of the computational logic could be shared between the ICD and the remote medicament concentration model and/or more sophisticated modeling could be achieved. Together, the result would be of benefit to the patient and to the caregivers that oversee the patient.

The focus on interaction implies a higher degree of user feedback during the day regarding their health status, and then using this input to drive a more sophisticated means of monitoring a person's adherence with higher confidence (for example, lower false positives and false negatives), and also going beyond this to help monitor well being (for example, monitoring for suicidal thoughts and providing rapid intervention in the event of untoward feedback) and even change behavior (for example, adjust doses within protocol limits based upon feedback such as pain scales).

This higher degree of user feedback can be gained through interactive sequences of questions through the ICD.

These interactive questions may comprise questions involving physiological state, psychological state or behavior. For example, “How much pain do you feel on a scale from 1 to 10, where 1 is no pain and 10 is very bad pain?” Or, for example, “How recent was your last meal—press 0 for less than one hour, or the number of hours.” Or, “what is your pulse?” Or, “are you being monitored now by a nurse to visually ensure that you took your medicine?”

In asking these interactive questions, one can see how they can be of value in better predicting a pharmacokinetic response, and therefore improve the confidence in the assessment of adherence. Those familiar with PK curves, know that different compounds are affected differently by fed/fasted states, high fat-content meals, glucose levels, etc. And, if a model is able to incorporate information such as the time since last meal, the size of the last meal, the sugariness of the last meal, it will be more apt to be able to judge whether a patient's blood level readings correlate to an adherent or non-adherent state.

For example, if a standard PK model anticipates a given range of blood concentration at 2.0 hours after administration, AND it is known that a patient had a recent large meal, then the range of blood concentration could be adjusted lower to account for the meal.

A situation for use of such inventions could be visualized with a hypothetical scenario of a patient we can call “Jim”. Jim has a psychological ailment that requires psycho-therapeutic drug treatment and is a patient in a government funded psych hospital and parole program. As part of this program, Jim is given a prescription for his drug, a supply of the drug, an ICD and an introductory lesson in using the ICD. He is told that he will receive free drug and be able to work outside the psych hospital as long as he is adherent, and may risk losing his freedom if he is not adherent. When he begins the program, he is still in residence in the psych hospital and each administration is monitored by a nurse to ensure adherence. He follows a standard protocol—each morning, Jim is given an alarm together with a text message “take one blue/orange capsule in the next hour, and dial *999 after you have taken your medicine”. Jim takes the medicine a half-hour later, and then dials *999 into the system. Two hours hence, an alarm sounds and a message “take sensor reading within the next hour, press *888 to start the test. 20 minutes later, Jim presses *888 and completes the sensor test (ie: saliva or breath test). The ICD asks a question: “Do you feel suicidal thoughts, press 1 for no or 9 for yes.” The ICD poses another question: “How many hours since your last meal?” After successfully using the system in monitored conditions for two weeks, the ICD and medicament concentration model have 14 data points that show Jim's PK readings are 10% higher than standard, and are impacted by up to 50% following a heavy meal. Jim is now released into the public and utilizes the system. The medicament concentration model now has a better way to establish anticipated tracer ranges for Jim's sensor results. The model now calculates the tracer concentration and analyzes it against Jim's custom expected PK for the specific time following ingestion and makes a judgment whether Jim is staying adherent. The results are displayed to Jim as a reminder to keep adherent and uploaded to a central network that monitors all of the patients of the psych hospital program.

And if Jim is non-adherent or reports suicidal thoughts, a caregiver places an immediate call to Jim to intervene.

### EXAMPLE 1

The ability to detect the blood concentration of a pain reliever, such as liquid acetaminophen in the blood stream by measuring a tracer is described. The research is carried out by two researchers A & B.

### Equipment Required:

10 ml liquid cough medicine (acetaminophen)
10 ml of 80 proof vodka

### Breathalyzer (Available as part of a retail cell phone in 2005 in Korea)

### Mass Spectrometer

### Study subject old enough to consume acetaminophen and alcohol

### Separate space so that “A” can be blind from “B”.

### Process:

Researcher “A” combines the liquid cough medicine together with the vodka in a common flask. “A” stirs until mixed. “A” gives the mixture to the study subject to consume orally. Waits 30 minutes. Subject breathes into breathalyzer, “A” extracts blood sample. “A” repeats both measurements 8 more times at 30 minute intervals. “A” compares alcohol readings with a PK curve for alcohol; compares mass spectrometer analysis of blood for the presence of acetaminophen with a PK curve for acetaminophen.

### Anticipated Thought Result:

We anticipate that “A” should be able to see that the PK results for both acetaminophen and alcohol fall within range of the standard PK curve.

### Continued:

The standard PK curves for alcohol & acetaminophen are provided to “B”, and he is told the must analyze one reading that was taken 2 hours after ingestion, and determine whether the alcohol reading was within the anticipated range of PK curve. He is given the alcohol breathalyzer result for 2 hours after ingestion.

We anticipate the “B” would find the 2-hour reading to fall within statistical range for alcohol.

And if the alcohol were thoroughly mixed with the acetaminophen, we could also assume “B” could predict the anticipated concentration of the acetaminophen for 2-hours after ingestion.

### Conclusion:

That combining a tracer with a medicine will yield predictable PK results over time for both compounds. Since both are predictable, they can be mathematically correlated. Because of this direct correlation, only one need be measured in order to predict the other. However, variation will exist in both models and be additive, therefore making predictions subject to statistical error. But, if there is trusted baseline data that was generated while the subject was closely monitored, one can adjust the standard PK curve for the tracer to a curve that is customize for the patient. By reducing the variation in the tracer, we can reduce the variation in the predicted medicament value.

### Characteristics of Suitable Detector/Analyzers:

As of 2005, alcohol breath sensor analyzers were commercially available as integral units within some retail cell phones sold in Korea. And while alcohol is not a suitable tracer analyte, one can infer from this commercialization of alcohol breathalyzer cell phones, that similarly enabled cell phones that sense other compounds should also be commercially viable.

The methods and processes shared in this disclosure will work with both portable ICD's as well as stand alone “table top” type of units that remain at a patient's home or within a central service facility such as a pharmacy or dispensary.

For purposes of simplicity, sensors in this document refer to devices which have the capability of sensing/detecting presence of an analyte in varying quantities and generating an apt signal (internal to the detector/analyzer) which is processed and analyzed through requisite circuitry to yield a signal (external to the detector/analyzer) that represents the results of the test. For simplicity's sake, the necessary internal signal conditioning, pre-processing, processing, post-processing, pattern recognition, buffering, counting and all other steps are assumed to be contained in the detector/analyzer unit. The ability to share processing power, circuitry, electrical power supply, user interface, etc with the host electronic device is fully anticipated by this inventor, but should be left to the discretion of the original equipment manufacturers and their suppliers.

Preferred ICD's should be sufficiently compact to enable it to be easily transportable and create minimal interference with the patient's daily life. This can be accomplished in many ways. In one embodiment, a breath analyzer could be integrated in close proximity to the microphone of an electronic device (eg: cell phone). In another embodiment, transdermal skin analyzers could be integrated with the touchpad or case of an electronic device (eg: cell phone or portable video game).

It should also be of reasonable cost, weight and power consumption. It must have a suitable and reliable way of communicating the results of the analysis with the host electronic device or a central monitoring facility.

Non-transportable-type stand alone type designs may also be considered that could be kept on a table or counter in a person's residence or at a local community facility (ie: pharmacy, nurse station, convenience store, post office, etc). The use of a central or home-based analyzer would enable the analysis to be performed and then an appropriate signal provided. This signal from the results of the analysis could be transmitted electronically (ie: plug-in download, wireless, RF, IR, PCM, etc.) to enable an electronic device for closed loop incentives (ie: cell phone minutes, songs, videos, game time, ring tunes, other software, etc), or it could be a simple diagnostic alphanumeric code that is then punched into the keypad of the patient's electronic device for incentives (ie: cell phone minutes, songs, videos, game time, etc), or the result could be transmitted directly to a central monitoring system (ie: over land lines or cellular phone network) for centrally based incentives (ie: food, water, baby food, etc).

The ICD should also have sufficient processing capability to manage reminder scheduling, display of information to inform patient what pills to take, detector/analyzer control, downstream logic associated with results, and historical record keeping. Host devices that have physical space to integrally house the detector/analyzer are ideal. Host devices that have physical space to house drug doses are also ideal. Host devices should have communication capability to a central monitoring station.

### Frequency of Analysis:

Analysis could be called for on a variety of schedules. In circumstances where a central community analyzer will be employed, analysis might only be possible several times each month. In circumstances where a home-based analyzer will be employed, analysis might be called for on a daily basis. In circumstances where an analyzer is integrated with the electronic device, and requires a special activity (ie: blowing through a breathalyzer tube), analysis might be called for more than once daily. In circumstances where an analyzer is integrated into an electronic device, and does not require special activity (ie: passive analysis of breath through an analyzer/detector in close proximity to the microphone of a cell phone), analysis might be made multiple times throughout a day.

### Addressing Drug & Tracer Formulation and Manufacture

A tracer analyte (or analytes) could be commingled with the active drug in many ways, for example in a similar dose form (ie: liquid with liquid, dry ingredient with dry ingredient, etc), or integrated into the coating (pills) or capsule (gelatin type integration), or other ways described later in the document. Components of the capsule, coating or colorant may act as a surrogate analyte.

A suitable tracer analyte must have known risk qualities and be considered safe by regulatory standards. Most foods, spices, and OTC monograph compounds are all examples that fall within this safety window. A suitable tracer analyte should not make patients feel bad (ie: no significant side effects). It should also be able to commingle with the active drug substance in a way that does not materially impact the active drug. It must also be sufficiently concentrated and prepared so as to minimize any added discomfort during ingestion (ie: not too bulky or bad tasting). It must also be detectable and analyzable by low cost and/or portable analyzers. It must also have a low likelihood of being found in normal environmental interactions of the patient population in study to prevent misleading readings from analytes found in food, air, work environments, etc. And if it normally occurs in the body, its presence should be able to be quantified so as to understand what percentage was naturally occurring and what percentage was related to the PK profile of the ingested combination.

The analyte should also have absorption, distribution, metabolism & excretion (ADME) qualities that lead to a predictable degradation and half-life in the body that facilitate appropriate analysis intervals (ie: half-life needs to be commensurate with the anticipated testing intervals and commensurate with the half-life of the medicament). In other words, if analysis is performed multiple times each day, the half-life would need to be a matter of hours to facilitate testing; if analysis is performed only a few times each month, half-life would need to be much longer in order to detect a longer period of drug consumption and best report compliance between analyses.

The PK profile of the tracer analyte provides an anticipated range of drug concentration according to time after ingestion. Knowing the PK profile and half-life of a drug or tracer analyte, one can make reasonable estimate of what would the blood concentration be anticipated any number of hours after taking a drug. One can also make a reasonable estimate of what the cumulative blood level concentration would be after subsequent days of taking the drug according to protocol.

Tracer analytes that are emitted in the breath from oral/throat surfaces (after contact with the tracer analyte) may also be used, but would not provide the same level of accuracy as analytes that emit from the blood. Analytes in the blood stream may be detected and analyzed in many ways. For example—breath testing, blood testing, skin testing, luminescence testing, saliva testing, etc. Preferred embodiments will use tests that are non invasive and which do not require exposure to blood. Breath testing of air passing near the aioli in the lungs will yield measurements that represent concentrations of commingled analytes within the blood stream and thus reflect patient consumption of the active drug.

### Possible Tracer/Sensor Pairs:

### Alcohol/Breathalyzers

There are a broad number of highly accurate, inexpensive breath alcohol analyzers. Their existence in cell-phones proves the validity of the technical reality of this invention. However, alcohol has too many negative human consequences to be considered for other than proof-of-concept studies.

### VOC's/Carbon Nanotube Sensors

Carbon nanotube sensors have received broad scientific press lately. They promise an ability to measure a broad range of analytes when they are commercialized in the not too distant future. However, they are not now commercially available.

### Volatile Sulfur Compunds (VSCS)

Such as diallyl disupfide (DADS) or Allyl mercaptan (2-propene-1-thiol). This is the most prevalent exhalent after garlic ingestion. After ingestion of garlic, several compounds can be detected in the breath, some immediately while others do not appear for several hours and then last for up to 30 hours. Lassko et al described the difference between the two batches of exhaled analytes due to the interaction of the garlic with the oral cavity (for immediately detected analytes) or with internal biological processes leading to excretion via the lungs (the compounds detected later). The drug could be supplemented with this compound as a tracer which would be assessed within minutes of ingesting the drug. Requires no metabolism by the body, but is exhaled directly from the oral cavity.

### Medium: Breath

DADS forms sulfur dioxide when exposed to oxygen. Sulfur dioxide detection is straight forward and there are small (hand-held), accurate methods to measure. A device that involved oxygenation of DADS could potentially be used.

DADS is a naturally occurring substance in bad breath, and individuals would not be able to eat garlic while under adherence monitoring

### Phytochemicals

There are a number of phytochemical classes that are GRAS (FDA designation as Generally Regarded As Safe). Many are used as spices in cooking, or in perfumes. These classes and examples are shown below:

### Terpenes:

### Monoterpene Apiacene family (cumin, fennel, caraway)

### Tetraterpenes Paprika, saffron, juniper, ginger, turmeric, galangal

### Terpene derivatives Coriander

### Phenylpropenoids

### Cinnaminic Cinnamon

### Eugenol Cloves

### Vanillin Vanilla Bean

### Diaryheptanoids

### Curcumin Turmeric

### Isothiocanates

### Allyl isothiocyanate Mustard seed, wasabi

6-metylsulfinylhexyl Wasabi
isothiocyanate
a) Cumin Extract:

eg cuminicum, cymene, dipentene, limonene, pinene. The compounds have a strong spicy smell.

They are terpenes which can be detected in the breath in small amounts by Selected Ion Flow Tube (SIFT) which allows analysis of complex mixtures.

### Sensor Medium: Breath or Urine

Detection methods: 1. SIFT has been used in the past to detect terpenes in the breath.

SIFT equipment would need to be at a pharmacy, clinic or lab at present, but could be arranged to provide near immediate results.

b) Alpha Pinene:

Alpha pinene has been detected in the urine of sawmill workers but not in normal urine. It is a component of cumin as stated in a)

### Sensor Medium: Breath

Detection method: Gas chromatography: The samples were collected and cleaned on a SEP-PAK cartridge and then analyzed with gas chromatography. After cleaning the samples were stable at 20 degrees C. for 12 weeks. Samples could be collected if the cleaning cartridge could be incorporated into the collection device and then stored for later analysis at a pharmacy (or clinic) every month. The PK of alpha pinene in humans is not known so the method would be qualitative until statistically defined.
c) Citral

Citral is a terpenoid found in lemongrass, and lemon scented oils. In rats 50% of the given dose is excreted in urine within 24 hours of administration. The fraction excreted in human urine is to be determined.

### Sensor Medium: URINE

Detection methods: It has been measured in the past by a calorimetric method using a Schiff reaction which is not quantitative unless calibrated using more traditional methods of detection such a gas chromatography.

PK is not clear in humans although many studies have looked at the affects of citral on the metabolism of other compounds. This would be a novel method if the technicalities could be worked out.

### Acetone:

Formed in vivo when there is metabolic stress present such as when the body uses fat for energy rather than glucose. Gives the breath a sweet smell that is characteristic of a diabetic crisis.

### Sensor Medium: Breath

Detection: multiple methods including a low cost, portable device that uses an optical method.

The breath is passed over a reactor filled with hydroxylamine (HA) which produces HCL which is then measured by near infrared diode laser spectroscopy.

Acetone may be a difficult tracer due to its normal presence in breath. Concentration may be used to differentiate but then care must be taken not to mask real metabolic deficits.

### Colorants/Dyes/Optical sensors

Flourescein dyes and indocyanide green dyes are commonly used in ophthalmic retina observations and are likely candidates to be measured in-situ through optical reflective/refractive technology.

### Caffeine/Immunoassay

Caffeine has been detected with an immunoassay film badge. This technique has not been validated for other chemicals but is a potential portable device to monitor drug compliance over a weekly period for example. This is the same principle as that use to measure radiation exposure over a period of time in a laboratory setting for example.

NOTE: Any of the above tracers could be used with the e-nose devise when it becomes cheap enough. There would need to be a period of collection of breath samples to distinguish ‘non-tracer’ breath from ‘tracer’ breath patterns.

Initial distribution of e-nose devices could reside at pharmacies or clinics, and patients could use the device in real time or bring in a sample or collection of samples when the refill their prescription.

What is claimed is:

1. A method of monitoring pharmaceutical adherence of a patient, comprising the steps of administering a medicament to the patient, acquiring data associated with administration of the medicament; transmitting the data associated with said administration to an interactive communication device; and displaying the data, wherein the display allows a determination of the pharmaceutical adherence of the patient to the medicament.

2. The method of claim 1, wherein the step of administration comprises administering a medicament having at least one active drug compound and at least one tracer compound and wherein the at least one tracer compound is capable of being detected by the interactive communication device.

3. The method of claim 1, further comprising the step of allowing the interactive communication device to transmit the data to a network server, wherein said server integrates the data into a medicament concentration model of the patient.

4. The method of claim 2, wherein the step of transmitting to an interactive communication device comprises a device having at least one sensor capable of detecting at least one tracer compound in a bodily fluid of the patient.

5. The method of claim 4, wherein the bodily fluid is selected from the group consisting of breath, saliva, and blood.

6. The method of claim 1, wherein the step of acquiring the data associated with administration of the medicament comprises acquiring data selected from the group consisting of physiological data, psychological data and behavioral data.

7. A method of creating an interaction between a patient and a medicament concentration model of a medicament administered to the patient, comprising the steps of: acquiring data associated with administration of the medicament to the patient with an interactive communication device, wherein said device is communicatively coupled to a network server; and allowing the network server to integrate the data into a drug concentration model of the medicament.

8. The method of claim 7, wherein the step of acquiring the data further comprises acquiring at least a portion of the data from the patient using an interactive sequence of questions through the interactive communication device so as to elicit at least one response from the patient.

9. The method of claim 8, wherein said step of acquiring the data comprises acquiring an interactive sequence of questions selected from the group consisting of questions associated with the patient's behavior, questions associated with the patients' psychology, and questions associated with the patient's physiology.

10. The method of claim 7, wherein the step of acquiring the data comprises acquiring data sufficient to predict a pharmacokinetic response of the patient to the medicament.

11. The method of claim 10, wherein the medicament administered to the patient comprises at least one active drug compound and at least one tracer compound.

12. The method of claim 11, wherein the step of acquiring data with an interactive communication device further comprises said acquiring data with said device that has at least one sensor for detecting at least one tracer compound in a bodily fluid of the patient.

13. The method of claim 12, wherein the step of allowing the server to integrate the data further comprises the steps of taking at least one sensor measurement of said at least one tracer compound and predicting at least one concentration of tracer compound in a fluid of the patient.

14. The method of claim 12, wherein the step of allowing the server to integrate the data further comprises the steps of taking at least one sensor measurement of said at least one tracer compound and predicting at least one concentration of active drug compound in a fluid of the patient.

15. The method of claim 10, the step of integrating data comprises utilizing at least one pre-existing pharmacokinetic model of said at least one active drug compound.

16. The method of claim 10, further comprising incorporating data sufficient to predict a pharmacokinetic response of the patient to the medicament into a behavior-modification system as a means to assist the user to maintain the defined medicament protocol.

17. The method of claim 10, further comprising incorporating data sufficient to predict a pharmacokinetic response of the patient to the medicament into a clinical trial management process to enables elimination of at least one user that is not properly following the defined medicament protocol.
